(19) United States
(12) Patent Application Publication
(10) Pub. No.: US 2010/0184769 A1
(43) Pub. Date: Jul. 22, 2010

(54) PDE INHIBITORS FOR THE TREATMENT OF HEARING IMPAIRMENT

(75) Inventor: Peter SANDNER, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/664,396

(22) PCT Filed: May 31, 2008
(86) PCT No.: PCT/EP2008/004351

§ 371 Date: Dec. 13, 2009

(30) Foreign Application Priority Data
Jun. 13, 2007 (EP) 07011535.7

(51) Int. Cl. A61K 31/53; C07D 487/04; A61P 27/16; C12Q 1/42

(52) U.S. Cl. 514/243; 544/184; 435/21

(57) ABSTRACT

The invention provides pharmacological compositions comprising PDE-5 inhibitors for the treatment of hearing impairment i.e. hearing loss and tinnitus. The invention also provides methods of screening for such PDE-5 inhibitors for use in the preparation of medicaments for the treatment of hearing impairment i.e. hearing loss and tinnitus.

### TECHNICAL FIELD OF THE INVENTION

The present invention relates to phosphodiesterases (PDEs) and the pharmacology of PDE inhibitors. More particularly, the invention relates to PDE-5 inhibitors and their use for preparation of medicaments for the treatment of Hearing Impairment, i.e. Hearing Loss and Tinnitus.

### BACKGROUND OF THE INVENTION

Hearing impairment, i.e. Hearing loss and Tinnitus are affecting more than 250 million patients worldwide and are therefore a very common diseases. Hearing impairment decrease the quality of life of patients dramatically and could currently not be treated adequately. Hearing loss is often categorized in conductive hearing loss, sensorineural hearing loss, and mixed hearing loss, which is a combination of conductive and sensorineural hearing loss. Conductive hearing loss results from impairment of the external or middle ear, i.e. caused by ear infections. Sensorineural hearing loss includes sensory hearing loss, caused by a cochlea disorder. Neural hearing loss, results from damage of the vestibulocochlear nerve. Most of the cases of hearing loss are sensorineural and caused by i.e. a damage or loss of hair cells in the cochlea. Tinnitus, defined as the perception of sound in the absence of an acoustic stimulus, is often associated with sensorineural hearing loss. The pathophysiology of tinnitus is not well understood. The causes of tinnitus could be similar to the causes of hearing loss, e.g., acoustic trauma, ototoxic drugs, and infections but also includes psychosocial and stress related factors. As noted above, tinnitus is also a symptom of Meniere's disease. Like sensorineural hearing loss, tinnitus is most commonly associated with the inner ear and it is very difficult to treat.

Currently, there are no clinically proven medications for the treatment of tinnitus and hearing loss (sensorineural and neural) and a medication would be very desirable.

### DISCLOSURE OF THE INVENTION

The term “hearing impairment” refers to a defect in the ability to perceive sound and includes partial hearing loss, complete hearing loss, deafness (complete or partial), The term tinnitus, refers to the perception of non-existent sounds. The hearing impairment may be due to hair cell or neuron damage, wherein the damage is caused by a genetic disorder, loud sounds, ototoxicity, or any other such stressor described in the application. Hearing impairment includes sensorineural hearing loss, conductive hearing loss, combination hearing loss, mild (between 25 and 40 dB), moderate (between 41 and 55 dB), moderately severe (between 56 and 70 dB), severe (between 71 and 90 dB), and profound (90 dB or greater) hearing loss, congenital hearing loss, pre-lingual and post-lingual hearing loss, unilateral (affecting one ear) and bilateral (affecting both ears) hearing loss, or any combination of these, i.e., sensorineural/severe/postlingual/bilateral.

Another aspect of the invention is the demonstration the PDE-5 inhibitor Vardenafil 10 mg/kg applied orally per gavage at a dose of 10 mg/kg showed significant prevention from acustic trauma (AT) and substantial recovery from AT. (table 1).

The invention provides PDE-5 inhibitors which are useful for the treatment of hearing impairment. In particular, compounds of the invention are Sildenafil (3-[2-ethoxy-5-(4-methylpiperazin-1-yl)sulfonyl-phenyl]-7-methy 1-9-propy 1-2.4.7.8-tetrabicyclo [4.3.0]nona-3,8,10-then-5-one), Tadalafil ((6R,12aR)-2,3,6,7,12,12a-Hexahydro-2-methyl-6-(3,4-methylene-dioxyphenyl) pyrazino(1′,2′:1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Udenafil 5-[2-propyloxy-5-(1-methyl-2-pyrrolidinylethylamidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxyphenyl)methyl]amino}-2-[(2S)-2-(hydroxymethyl)pyrrolidin-1-yl]-N-(pyrimidin-2-ylmethyl)pyrimidine-5-carboxamide, SLx 2101 of Surface Logix, LAS 34179 Triazolo[1,2-]xanthine,6-methyl-4-propyl-2-[2-propoxy-5-(4-methylpiperazino)sulfonyl]phenyl-.

Still another aspect of the invention is a method of screening for PDE inhibitors, in particular for inhibitors of PDE-5 for use for the preparation of medicaments for the treatment of hearing impairment as defined above.

The invention provides methods (also referred to herein as “screening assays”) for identifying PDE inhibitors which can be used for the treatment of hearing disorders. The methods entail the identification of candidate or test compounds or agents (e.g., peptides, peptidomimetics, small molecules or other molecules) which bind to phosphodiesterases and/or have a stimulatory or inhibitory effect on the biological activity of PDE5 or its expression and then determining which of these compounds have an effect on symptoms or diseases regarding hearing impairment in an in vivo assay.

Candidate or test compounds or agents which bind to PDE-5 and/or have a stimulatory or inhibitory effect on the activity or the expression of PDE-5 are identified either in assays that employ cells which express PDE-5 (cell-based assays) or in assays with isolated PDE-5 (cell-free assays). The various assays can employ a variety of variants of PDEs (e. g., full-length PDEs, a biologically active fragment of PDEs, or a fusion protein which includes all or a portion of PDEs). Moreover, PDE-5 can be derived from any suitable mammalian species. The assay can be a binding assay entailing direct or indirect measurement of the binding of a test compound or a known PDE-5 ligand to PDE-5. The assay can also be an activity assay entailing direct or indirect measurement of the activity of PDE-5. The assay can also be an expression assay entailing direct or indirect measurement of the expression of PDE-5 mRNA and PDE-5 protein. The various screening assays are combined with an in vivo assay entailing measuring the effect of the test compound on the symptoms of hearing impairment.

The present invention includes biochemical, cell free assays that allow the identification of inhibitors and agonists of PDEs suitable as lead structures for pharmacological drug development. Such assays involve PDE-5 with a test compound and determining the ability of the test compound to act as an antagonist (preferably) or an agonist of the enzymatic activity of PDE-5. In one embodiment, the assay includes monitoring the PDE activity of PDE-5 by measuring the conversion of either cP or cGMP to its nucleoside monophosphate after contacting PDE-5 with a test compound.

For example, cAMP and cGMP levels can be measured by the use of the tritium containing compounds 3HcAMP and 3HcGMP as described in [Hansen, R. S., and Beavo, J. A., PITAS USA1982,79: 2788-92]. To screen a compound pool comprised of a large number of compounds, the microtiter plate-based scintillation proximity assay (SPA) as described in [Bardelle, C. et al. (1999) Anal. Biochem. 275: 148-155] can be applied.

Alternatively, the phosphodiesterase activity of the recombinant protein can be assayed using a commercially available SPA kit (Amersham Pharmacia). The PDE enzyme hydrolyzes cyclic nucleotides, e.g. cAMP and cGMP to their linear counterparts. The SPA assay utilizes the tritiated cyclic nucleotides [3H]cAMP or [3H]cGMP, and is based upon the selective interaction of the tritiated non cyclic product with the SPA beads whereas the cyclic substrates are not effectively binding.

Radiolabelled product bound to the scintillation beads generates light that can be analyzed in a scintillation counter.

A pharmaceutical composition of the invention is formulated to be compatible with its intended route of administration. Examples of routes of administration include parenteral e.g., intravenous, intradermal, subcutaneous’ oral (e.g. ‘inhalation)’ transdermal (topical) transmucosal and rectal administration. Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents for example, parabens, chlorobutanol, phenol, ascorbic acid, thimerosal, and the like. In many cases, it will be preferable to include isotonic agents, for example, sugars, polyalcohols such as maitol sorbitol sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or con1 starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g. ‘a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Bio degradable, biocompatible polymers can be used, such as ethylene vinyl acetate, polyanhydrides, polyglycolic acid, collagen, polyorthoesters, and polylactic acid.

The present invention provides further:

A method of screening for PDE 5 inhibitors useful as therapeutic agents in the treatment of hearing impairment referring to a defect in the ability to perceive sound including partial hearing loss, complete hearing loss, deafness (complete or partial), and tinnitus.

Methods of screening which involve contacting the test compound in or at the surface of a cell, wherein the cell is in vitro.

Methods of screening which involve contacting the test compound with the PDE-5 polypeptide in a cell free system.

Methods of screening may involve a test compound which is coupled to a detectable label.

In particular, the present invention provides:

A pharmaceutical composition for the treatment of a disease comprised in a group of diseases consisting of a consisting of hearing impairment, referring to a defect in the ability to perceive sound including partial hearing loss, complete hearing loss, deafness (complete or partial), and tinnitus.

Hearing impairment, referring to a defect in the ability to perceive sound including partial hearing loss, complete hearing loss, deafness (complete or partial), and tinnitus, in a mammal, comprising a therapeutic agent which regulates the activity of a PDE5 polypeptide.

A pharmaceutical composition for the treatment of a disease comprised in a group of diseases consisting of hearing impairment referring to a defect in the ability to perceive sound including partial hearing loss, complete hearing loss, deafness (complete or partial), and tinnitus in a mammal comprising a PDE-5 inhibitor selected from the group of PDE-5 Inhibitors consisting of Sildenafil (3-[2-ethoxy-5-(4-methylpiperazin-1-yl)sulfonyl-phenyl]-7-methy 1-9-propy 1-2.4.7.8-tetrabicyclo [4.3.0]nona-3,8,10-trien-5-one), Tadalafil ((6R,12aR)-2,3,6,7,12,12a-Hexahydro-2-methyl-6-(3,4-methylene-dioxyphenyl) pyrazino(1′,2′:1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Udenafil 5-[2-propyloxy-5-(1-methyl-2-pyrrolidinyl-ethyl-amidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S)-2-(hydroxymethyl)pyrrolidin-1-yl]-N-(pyrimidin-2-yl methyl)pyrimidine-5-carboxamide, SLx 2101 of Surface Logix, LAS 34179Triazolo[1,2-]xanthine,6-methyl-4-propyl-2-[2-propoxy-5-(4-methylpiperazino)-sulfonyl]phenyl or salts, hydrates or hydrates of salts thereof.

Use of a PDE5 inhibitor for the preparation of a pharmaceutical composition for the treatment of a disease comprised in a group of diseases consisting of hearing impairment referring to a defect in the ability to perceive sound including partial hearing loss, complete hearing loss, deafness (complete or partial), and tinnitus in a mammal.

Use of PDE-5 inhibitor selected from the group of PDE-5 Inhibitors consisting of Sildenafil (3-[2-ethoxy-5-(4-methylpiperazin-1-yl)sulfonyl-phenyl]-7-methy 1-9-propy 1-2.4.7.8-tetrabicyclo [4.3.0]nona-3,8,10-trien-5-one), Tadalafil ((6R,12aR)-2,3,6,7,12,12a-Hexahydro-2-methyl-6-(3,4-methylene-dioxyphenyl) pyrazino(1′,2′:1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Udenafil 5-[2-propyloxy-5-(1-methyl-2-pyrrolidinyl-ethyl-amidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S)-2-(hydroxymethyl)pyrrolidin-1-yl]-N-(pyrimidin-2-yl methyl)pyrimidine-5-carboxamide, SLx 2101 of Surface Logix, LAS 34179Triazolo[1,2-]xanthine,6-methyl-4-propyl-2-[2-propoxy-5-(4-methylpiperazino)-sulfonyl]phenyl or salts, hydrates or hydrates of salts thereof, for the preparation of a pharmaceutical composition for the treatment of a disease comprised in a group of diseases consisting of hearing impairment referring to a defect in the ability to perceive sound including partial hearing loss, complete hearing loss, deafness (complete or partial), and tinnitus in a mammal.

A method for the preparation of a pharmaceutical composition wherein the inhibitor of PDE5 is a PDE-5 inhibitor selected from the group of PDE-5 Inhibitors consisting of Sildenafil (3-[2-ethoxy-5-(4-methylpiperazin-1-yl)sulfonyl-phenyl]-7-methy 1-9-propy 1-2.4.7.8-tetrabicyclo [4.3.0]nona-3,8,10-trien-5-one), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Tadalafil ((6R,12aR)-2,3,6,7,12,12a-Hexahydro-2-methyl-6-(3,4-methylenedioxyphenyl), Udenafil 5-[2-propyloxy-5-(1-methyl-2-pyrrolidinyl-ethyl-amidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S)-2-(hydroxymethyl)pyrrolidin-1-yl]-N-(pyrimidin-2-yl methyl)pyrimidine-5-carboxamide, SLx 2101 of Surface Logix and LAS 34179Triazolo[1,2-]xanthine,6-methyl-4-propyl-2-[2-propoxy-5-(4-methylpiperazino)-sulfonyl]phenyl.

Use of a pharmaceutical composition as mentioned above for the regulation of PDE activity in a mammal having a disease comprised in a group of diseases consisting of hearing impairment referring to a defect in the ability to perceive sound including partial hearing loss, complete hearing loss, deafness (complete or partial), and tinnitus.

A preferred embodiment of the invention is a pharmaceutical composition containing Vardenafil, or a salt, a hydrat or a hydrat of a salt thereof, for the treatment of a disease comprised in a group of diseases consisting of hearing impairment referring to a defect in the ability to perceive sound including partial hearing loss, complete hearing loss, deafness (complete or partial), and tinnitus in a mammal.

### DESCRIPTION OF FIGURES AND TABLES

TABLE 1
Table 1: Effect of Vardenafil and Placebo treatment on
acustic trauma and threshold levels of rats.
Hearing Threshold in [dB SPL]

| Measurment | Placebo | Vardenafil |
|---|---|---|
| A | 7.1 | 5.9 |
| B | 83.8 | 38.6 |
| C | 80.8 | 28.3 |
| D | 66 | 19.5 |
| E | 57.1 | 16.5 |
| F | 53.4 | 14.3 |
| G | 49 | 12.4 |
| H | 42.8 | 11.2 |
| I | 43.1 | 10.9 |
| J | 44.6 | 8.1 |

### EXAMPLE 1

All animal experiments were performed due to the “German Law for the Protection of Laboratory animals” and were conducted due to the approved guidelines for Animal Health and Welfare. Experiments were performed with female Sprague Dawley Rats with a body weight between 300-400 g. For induction of acoustic trauma (AT) animals were kept under anesthezia (Ketamine, Xylazin, Rompun i.p. injection) and exposed to band noise or pure tones using a calibrated loudspeaker inside a reverberating chamber. The sound consists of a continuous 10 kHz pure-tone presented at 115 dB SPL. All acoustic stimuli were calibrated at the head level of the animal. Rats were treated with either Vardenafil [10 mg/kg/ p.o. dissolved in Ethanol/Solutol/Water (10/40/50) with an application volume of 5 ml/kg] or Placebo [Ethanol/Solutol/Water (10/40/50) with an application volume of 5 ml/kg] twice daily. The first treatment with Vardenafil or Placebo was i.e. 1 h prior to AT. The development and progression/remission of hearing impairment was detected by measuring the hearing thresholds by recordings of auditory brainstem responses (ABR). The threshold was determined by the lowest sound pressure that produced ARBs distinct from noise level. Threshold level analysis was performed prior to the acustic trauma (AT) (Measurement A in table 1), 3-5 hours post AT (Measurement B in table 1), once daily, from day 1 to day 7 post AT (Measurement C to I in table 1) and finally 3 weeks post AT (Measurement J in table 1).

Prior to AT, our results showed threshold levels in the Levitra treated group and in the Placebo-treated group of 5.9 dB SPL and 7.1 dB SPL respectively. These levels were not significantly different between the groups and in the physiological range of hearing thresholds in the rat. AT significantly increased the hearing threshold in Placebo-treated animals to 83.8. and 38.6 in placebo- and vardenafil-treated animals respectively. These results indicate that Vardenafil treatment prevented from hearing loss. Moreover in the vardenafil treated group there was complete remission of the hearing loss after day 5 of Vardenafil treatment. Vardenafil-treated animals showed a threshold level of 12.4 which was not significantly different from the prae AT threshold level in this group. Placebo-treated animals could not recover from acustic trauma. These results strongly suggest that PDE5 inhibitors, i.e. Vardenafil could prevent hearing impairment.

1. A method of screening for PDE 5 inhibitors useful as therapeutic agents in the treatment of a disease comprised in a group of diseases consisting of “hearing impairment” referring to a defect in the ability to perceive sound including partial hearing loss, complete hearing loss, deafness (complete or partial), and tinnitus comprising the steps of i) contacting a test compound with a PDE5 polypeptide, ii) determining the activity of the PDE5 polypeptide at a certain concentration of the test compound or in the absence of said test compound, iii) determining the activity of said PDE5 polypeptide at a different concentration of said test compound, iv) selecting at least one compound with inhibitory effect on the PDE-5 polypeptide.

2. A pharmaceutical composition for the treatment of a disease comprised in a group of diseases consisting of “hearing impairment” referring to a defect in the ability to perceive sound including partial hearing loss, complete hearing loss, deafness (complete or partial), and tinnitus in a mammal comprising a therapeutic agent which regulates the activity of a PDE5 polypeptide.

3. A pharmaceutical composition for the treatment of a disease comprised in a group of diseases consisting of “hearing impairment” referring to a defect in the ability to perceive sound including partial hearing loss, complete hearing loss, deafness (complete or partial), and tinnitus in a mammal comprising a PDE-5 inhibitor selected from the group of PDE-5 Inhibitors consisting of Vardenafil, Sildenafil, Tadalafil, Udenafil, Dasantafil, Avanafil, SLx2101 and LAS34179.

4. Use of PDE-5 inhibitor selected from the group of PDE-5 Inhibitors consisting of Vardenafil, Sildenafil, Tadalafil, Udenafil, Dasantafil, Avanafil, SLx2101 and LAS34179 for the preparation of a pharmaceutical composition for the treatment of a disease comprised in a group of diseases consisting of “hearing impairment” referring to a defect in the ability to perceive sound including partial hearing loss, complete hearing loss, deafness (complete or partial), and tinnitus in a mammal.

5. A pharmaceutical composition containing at least on compound selected from the group Vardenafil, Sildenafil, Tadalafil, Udenafil, Dasantafil, Avanafil, SLx2101 and LAS34179 or a salt, a hydrat or a hydrat of a salt thereof, for the treatment of a disease comprised in a group of diseases consisting of “hearing impairment” referring to a defect in the ability to perceive sound including partial hearing loss, complete hearing loss, deafness (complete or partial), and tinnitus in a mammal.

6. A pharmaceutical composition containing at least one compound selected from the group Vardenafil, Sildenafil, Tadalafil, Udenafil, Dasantafil, Avanafil, SLx2101 and LAS34179 or a salt, a hydrat or a hydrat of a salt thereof, for the treatment of hearing loss and tinnitus.

7. A pharmaceutical composition containing Vardenafil or a salt, a hydrat or a hydrat of a salt thereof, for the treatment of hearing loss and tinnitus.

8. Use of at least one compound selected from the group Vardenafil, Sildenafil, Tadalafil, Udenafil, Dasantafil, Avanafil, SLx2101 and LAS34179 or a salt, a hydrat or a hydrat of a salt thereof, for the preparation of a pharmaceutical composition for the treatment of hearing loss and tinnitus.

9. Use of Vardenafil or a salt, a hydrat or a hydrat of a salt thereof, for the preparation of a pharmaceutical composition for the treatment of hearing loss and tinnitus.
